(19) United States
(12) Patent Application Publication
(10) Pub. No.: US 2016/0109430 A1
(43) Pub. Date: Apr. 21, 2016

(54) METHODS FOR DRUG DISCOVERY

(71) Applicant: Memorial Sloan-Kettering Cancer Center, New York, NY (US)

(72) Inventors: ANDREW KOFF, Westbury, NY (US); Marta Kovatcheva, New York, NY (US); Mary Elizabeth Klein, New York, NY (US)

(73) Assignee: Memorial Sloan-Kettering Cancer Center, New York, NY (US)

(21) Appl. No.: 14/919,505

(22) Filed: Oct. 21, 2015

Related U.S. Application Data
(60) Provisional application No. 62/167,152, filed on May 27, 2015.
(60) Provisional application No. 62/066,779, filed on Oct. 21, 2014.

(51) Int. Cl. G01N 33/50

(52) U.S. Cl. CPC G01N 33/5023; G01N 2333/914; G01N 2500/10

(57) ABSTRACT

The present invention provides assays for identifying drug candidates that regulate cellular senescence. In particular, the present invention relates to the use of ATRX foci as a biomarker for determining whether one or more drug candidates regulate senescence in a cell line. In one non-limiting embodiment, the present invention provides assays to identify compounds that can be used in a combinatorial cancer treatment. The present invention is based, at least in part, on the discovery that the number of ATRX foci increases in cells that undergo senescence. Accordingly, in non-limiting embodiments, the present invention provides for assays and kits for identifying drug combinations that may be useful in treating subject that have cancer and for identifying compounds that may be useful in treating age-related diseases.

### CROSS-REFERENCE TO RELATED APPLICATIONS

This application claims priority to U.S. Provisional Application Ser. No. 62/066,779, filed Oct. 21, 2014, and U.S. Provisional Application Ser. No. 62/167,152, filed May 27, 2015, to each of which priority is claimed and the contents of each which are incorporated herein in their entireties.

### GRANT INFORMATION

This invention was made with government support under Grant No. P50CA 140146 awarded by the National Institutes of Health. The government has certain rights in the invention.

### I. INTRODUCTION

This present invention relates to ATRX foci as a biomarker for determining whether cells are undergoing senescence. In particular, ATRX foci can be used as a biomarker to identify drug candidates for use in combinatorial cancer treatments and/or as a biomarker for identifying compounds that prevent or inhibit senescence. As such, evaluating ATRX foci may be used as part of a drug discovery process.

### 2. BACKGROUND OF THE INVENTION

Alpha-thalassemialmental retardation syndrome X-linked (ATRX) is encoded by the atrx gene. ATRX is a SWI/SNF helicase/ATPase that can regulate gene expression via chromatin remodeling and is associated with pericentric and telomeric heterochromatin (McDowell et al. PNAS 1999; Eusterrnann et al. NSMB 2011). Its primary clinical indication is mutations in the mental retardation syndrome α-thalassemia/MR, X-linked (ATRX syndrome) (Picketts D J et al. Am. J. Human Genet. 1996).

Although ATRX can interact with several proteins that are involved in senescence including PML bodies (Xue et al. PNAS 2003; Luciani et al. J. Cell Science 2006), HP1 proteins (McDowell et al. PNAS 1999; Eustermann et al. NSMB 2011) and macroH2A (Ratnakumar et al. Genes and Dev 2012), ATRX has never been directly associated with senescence, and studies have shown that ATRX negatively regulates macroH2A (a facilitator of senescence-associated heterochromatic foci formation) incorporation into chromatin (Ratnakumar et al. Genes and Dev 2012).

There is a link between age-related pathologies and senescence. The appearance of senescence cells in premature aging skin can lead to age-related dermal and epidermal thickening and loss of collage (Waaijer et al. Aging Cell 2012). Senescence in astrocytes can promote age-related neurodegeneration giving rise to cognitive impairment and contribute to Alzheimer's and Parkinson's disease (Bitto et al. Exp. Cell Res. 2010). Senescent chondrocytes play a role in osteoarthritis (Roberts et al. Eur. Spine J. 2006) and senescent endothelial cells and smooth muscle cells may contribute to atherosclerosis (Matthews et al. Circ. Res. 2006). A causal link between senescence and aging was first established in a progeroid mouse model in which p16-positive senescence cells could be eliminated early in mouse life and even later in life and many age-related dysfunctions were delayed by the elimination of these senescent cells (Baker et al. Nature 2012).

Efforts towards drug discovery continue to use vast technical and financial resources to identify and develop new and useful drugs. However, the identification of new and useful drug therapies for cancer and age-related diseases and pathologies continues to be difficult. The development of more effective cancer therapies is important for increasing a patient's survival and can improve treatment success. Furthermore, a therapy for treating age-related diseases can affect a person's quality of life and can delay the onset of such diseases. Therefore, there remains a need in the art for assays of identifying compounds that may be effective in such treatment regimens.

### 3. SUMMARY OF THE INVENTION

The present invention relates to assays and compositions for identifying compounds that regulate cellular senescence. In particular, the disclosed assays relate to the use of ATRX foci as a biomarker for identifying drug candidates that induce senescence or inhibit and/or reduce senescence in a cell line. In certain embodiments, the present invention provides assays and compositions that use ATRX foci as a biomarker to identify drug candidates for use in treating age-related diseases. In certain embodiments, the present invention related to assays that use ATRX foci as a biomarker to identify drug candidates for use in cancer treatments and, in particular, for identifying combinations of drugs for use in cancer therapy. The present invention is based, at least in part, on the discovery that the number of ATRX foci increases in cells that undergo senescence.

### 4. BRIEF DESCRIPTION OF THE FIGURES

FIG. 1 depicts a schematic representation of an assay according to a non-limiting embodiment of the present invention.

FIG. 2 shows that ATRX is required for senescence induced by the inhibition of CDK4 by PD0332991.

FIG. 3 shows that the number of ATRX foci increased upon treatment with doxorubicin as detected by immunofluorescence. Representative immunofluorescence images are shown on top and the mean and standard deviation of the foci counts from at least 50 cells in each of three independent experiments are plotted below (*p<0.01).

FIG. 4 shows LS8817 cells expressing either a scrambled (shSCR) or ATRX (shATRX) shRNA that were treated with 100 μM doxorubicin for five days and the effect on the accumulation of senescence associated β-galactosidase (β-gal) positive cells and expression of p53 and ATRX were determined. This experiment was repeated twice with independent transductants.

FIG. 5A-C shows that replicative senescence of untransformed cells is associated with an increase in the number of ATRX foci per cell. (A) Expression of phosphorylated Rb, a marker of cell proliferation, decreased upon passaging the cells to a high passage number. At passage 25, accumulation of perinuclear associated β-galactosidase (SA-β-gal) was observed (B) and the number of ATRX foci per cell significantly increased at passage 25 as compared to cells at passage 11 (C).

FIG. 6A-C show that the treatment of cells with PD0332991 followed by shMDM2 resulted in a significant increase in the number of ATRX foci within cells that do not respond to PD0332991 treatment compared to cells that were not treated with PD0332991 or were treated with PD0332991 alone. (A) shMDM2 expression resulted in a decrease in MDM2 expression. (B) Treatment of cells with PD0332991 followed by shMDM2 resulted in the accumulation of SA-β-gal. (C) Treatment of cells with PD0332991 followed by shMDM2 resulted in an increase in the number of ATRX foci per cell.

FIG. 7A-C shows that the expression of Flag-MDM2 blocks CDK4 inhibition-induced senescence. (A) Schematic of the tet-on-MDM2-Flag construct and the experimental conditions. (B) Treatment of cells with PD0332991 in the presence of exogenous MDM2 prevented an increase in the number of ATRX foci per cell compared to treatment with PD0332991 alone. (C) Treatment of LS8817 cells with 10 μM doxycycline to induce MDM2 expression (10 μM Dox), treatment with 10 μM doxycycline for 2 days to induce MDM2 expression followed by the addition 0.1 μM PD0332991 in the presence of doxycycline for an additional 7 days (0.1 μM PD/10 Dox), treatment with 10 μM doxycycline for 2 days to induce MDM2 expression followed by the addition of 0.1 μM PD0332991 in the presence of doxycycline for 2 days to arrest the cells in quiescence and the removal of doxycycline while treating the cells with 0.1 μM PD0332991 alone for another 5 days (0.1 μM PD/−Dox), or treatment with 0.1 μM PD0332991 for 7 days (0.1 μM PD).

FIG. 8A-C shows that the reduction in MDM2 expression induces senescence. (A) Schematics of the tet-on-shMDM2 constructs and the experimental conditions. (B) Treatment of LS8817 cells with doxycycline to induce expression of shMDM2 resulted in an increase in the average number of ATRX foci per cell compared to the control. (C) Treatment of LS8313 cells with doxycycline for 5 days to induce expression of shMDM2 resulted in an increase in the number of ATRX foci per cell as compared to the control.

### 5. DETAILED DESCRIPTION OF THE INVENTION

For clarity and not by way of limitation the detailed description of the invention is divided into the following subsections:

(i) ATRX foci as a biomarker;

(ii) methods of use; and

(iii) kits.

### 5.1 Atrx Foci as a Biomarker

Alpha-thalassemia/mental retardation syndrome X-linked is denoted ATRX herein.

The present invention discloses ATRX foci as a biomarker for senescence. The term “ATRX foci,” as used herein, refers to ATRX-positive punctate structures that can be visualized within a cell.

In a specific, non-limiting embodiment, ATRX foci may be detected using an immunodetection reagent specific for an ATRX protein.

In a specific, non-limiting embodiment, an ATRX protein may be a human ATRX protein having the amino acid sequence as set forth in NCBI database accession no. NP_000480.

ATRX proteins for non-human species are known or can be determined according to methods known in the art, for example, where the sequence is the allele represented in the majority of the population.

In a specific, non-limiting embodiment, an ATRX protein may be a mouse ATRX protein having the amino acid sequence as set forth in NCBI database accession no. NP_033556.

In a specific, non-limiting embodiment, an ATRX protein may be a rat ATRX protein having the amino acid sequence as set forth in NCBI database accession no. XP_003754859.

Methods for detecting and/or determining the number of ATRX foci can include, but are not limited to, immunofluorescence and immunoglobulin-mediated assays, and other techniques known in the art.

In certain, non-limiting embodiments, immunohistochemistry can be used for detecting ATRX foci. For example, an antibody that binds ATRX (“ATRX-specific antibody”) or a fragment thereof can be brought into contact with, for example, a thin layer of cells, followed by washing to remove unbound antibody, and then contacted with a second, labeled antibody, e.g., secondary antibody. Labeling can be by fluorescent markers, enzymes, such as peroxidase, avidin or radiolabeling. The labeling can be scored visually using microscopy and the results can be quantitated.

In non-limiting embodiments, methods of ATRX detection may utilize the ATRX-specific antibody sold by Bethyl, Catalog No. A301-045A (“the '045 Ab”), a fragment thereof, or an antibody that competitively inhibits binding of the '045 Ab to ATRX. In certain non-limiting embodiments, methods of ATRX detection may utilize the ATRX-specific antibody sold by Santa Cruz Biotechnology, Catalog No. sc-55584 (the “D5 antibody”), a fragment thereof, or an antibody that competitively inhibits binding of the D5 antibody to ATRX.

Non-limiting examples of antibody fragments include Fab, Fab′, F(ab′)₂, Fv, single chain Fv (scFv) or a variable region comprised in a chimeric molecule.

### 5.2 Methods of Use

The present invention relates to assays for identifying compounds that regulate cellular senescence by analyzing ATRX foci as a biomarker. As discussed in the Examples section below, senescence in cancer cells and untransformed cells correlates with an increase in the number of ATRX foci in each cell, and disruption of ATRX prevents senescence.

“Senescence,” as used herein, refers to a cell state in which the cell has little or no proliferative capacity as compared to quiescence, where a cell retains the capacity for proliferation. In certain embodiments, “senescence” or a “senescent state” refers to a cellular state where an increase in the expression of at least one marker, or at least two markers, or at least three markers of the senescent phenotype selected from the group consisting of SA-β-gal, senescence-associated heterochromatin foci and elaboration of the senescence-associated secretory program is observed in the cell.

In certain embodiments, the present invention provides for assays for identifying drug compounds that induce senescence. In certain embodiments, such drug compounds can be used in combination with other cancer agents as a cancer therapy.

In certain embodiments, the present invention further provides for assays that can be used for identifying compounds that prevent, inhibit, minimize and/or reduce senescence. In certain embodiments, such drug compounds can be used to prevent, minimize, inhibit and/or reduce senescence induced by aging and can be used to treat age-related diseases.

In certain embodiments, the disclosed assays can be used to screen large libraries of compounds. In non-limiting embodiments, the assays of the disclosed invention can be used to prioritize large numbers of new compounds for further drug development and/or can identify new compounds that can be used in combination with compounds currently being used clinically.

Candidate compounds (also referred to herein as drug candidates) to be screened in the currently disclosed assays include pharmacologic agents already known in the art as well as compounds previously unknown to have any pharmacological activity. One non-limiting example of a library that includes compounds that can be screened using the disclosed assays is an FDA approved library of compounds that can be used by humans. Synthetic compound libraries are commercially available from a number of companies including Maybridge Chemical Co. (Trevillet, Cornwall, UK), Comgenex (Princeton, N.J.), Brandon Associates (Merrimack, N.H.) and Microsource (New Milford, Conn.), and a rare chemical library is available from Aldrich (Milwaukee, Wis.). Alternatively or additionally, libraries of natural compounds in the form of bacterial, fungal, plant and animal extracts can be used in the disclosed assay, for example, from Pan Laboratories or MycoSearch. For example, and not by way of limitation, the drug candidates can include medicaments, vitamins, mineral supplements, substances used for the treatment and/or prevention of cancer or prodrugs, which become biologically active or more active after they have been placed in a physiological environment. Additional non-limiting examples of drug candidates include small molecules, antibiotics, antivirals, antifungals, enediynes, heavy metal complexes, hormone antagonists, non-specific (non-antibody) proteins, sugar oligomers, aptamers, oligonucleotides (e.g., antisense oligonucleotides that bind to a target nucleic acid sequence (e.g., mRNA sequence)), siRNA, shRNA, peptides, proteins, radionuclides, and transcription-based pharmaceuticals. In non-limiting embodiments, potential drug candidates can include nucleic acids, peptides, small molecule compounds (e.g., pharmaceutical compounds), and peptidomimetics. Candidate compounds can be naturally occurring compounds or synthetic compounds. For example, and not by way of limitation, the candidate compounds can be isolated from microorganisms, animals or plants, or can be produced recombinantly or synthesized by chemical methods known in the art.

The assays of the present invention may be performed in multiwell formats, in microtiter plates, in multispot formats or in arrays. In certain non-limiting embodiments, the cells for use in the present invention can be cultured and grown in 96-well microtiter plates. In certain non-limiting embodiments, the cells for use in the present invention can be cultured and grown in 384-well microtiter plates.

In certain, non-limiting embodiments, immunohistochemistry can be used for detecting ATRX foci in the presently disclosed methods. For example, an ATRX-specific antibody can be brought into contact with, for example, a thin layer of cells, followed by washing to remove unbound antibody, and then contacted with a second, labeled, antibody, e.g., secondary antibody. Labeling can be by fluorescent markers, e.g., fluorophores, enzymes, such as peroxidase, avidin or radiolabeling. Alternatively, or additionally, an ATRX-specific antibody that is conjugated to a fluorophore can be brought into contact with the cells, followed by washing to remove unbound antibody, without the need for a second, labeled antibody. Non-limiting examples of fluorophores include rare earth chelates or fluorescein and its derivatives, rhodamine and its derivatives, dansyl, umbelliferone, luciferases, e.g., firefly luciferase and bacterial luciferase (see U.S. Pat. No. 4,737,456, incorporated by reference herein in its entirety) and luciferin. The number of ATRX foci per cell can be scored visually using microscopy and the results can be quantitated. In a specific non-limiting embodiment, the '045 Ab, or an antibody that competitively inhibits binding of the '045 Ab to ATRX, can be used to detect ATRX foci. In a specific non-limiting embodiment, the D5 antibody, or an antibody that competitively inhibits binding of the D5 antibody to ATRX, can be used to detect ATRX foci.

In certain embodiments, an antibody that competitively inhibits binding of the '045 Ab or the D5 antibody to ATRX refers to an antibody that blocks binding of the '045 Ab or the D5 antibody to ATRX in a competition assay by about 50% or more, e.g., about 55% or more, about 60% or more, about 65% or more, about 70% or more, about 75% or more, about 80% or more, about 85% or more, about 90% or more, about 95% or more, about 98% or more or about 99% or more, and conversely, the '045 Ab or the D5 antibody blocks binding of the antibody to ATRX in a competition assay by about 50% or more, e.g., about 55% or more, about 60% or more, about 65% or more, about 70% or more, about 75% or more, about 80% or more, about 85% or more, about 90% or more, about 95% or more, about 98% or more or about 99% or more. An exemplary competition assay is described in “Antibodies,” Harlow and Lane (Cold Spring Harbor Press, Cold Spring Harbor, N.Y.)(1988).

### 5.2.1. Assay for Identifying Compounds that Induce Senescence

The present invention further assays for identifying drug candidates that can be used in a combinatorial cancer treatment. In particular, the present invention provides assays for identifying potential drug candidates that can be used to induce senescence in a cancer cell line. In certain embodiments, the assays of the present invention comprise analyzing the number of ATRX foci per cell observed in the cancer line following treatment with the drug combination.

The present invention provides assays for identifying drug candidates that may be effective as therapeutic agents for treating cancer early in the drug development and discovery process. Non-limiting examples of such cancers include soft tissue sarcomas, melanoma, breast cancer, lung cancer, liposarcoma, basal cell carcinoma and glioma (or glioblastoma). In non-limiting embodiments, the assays of the disclosed invention can be used to prioritize large numbers of new compounds for further drug development and/or can identify new compounds that can be used in combination with cancer agents that are currently being used clinically.

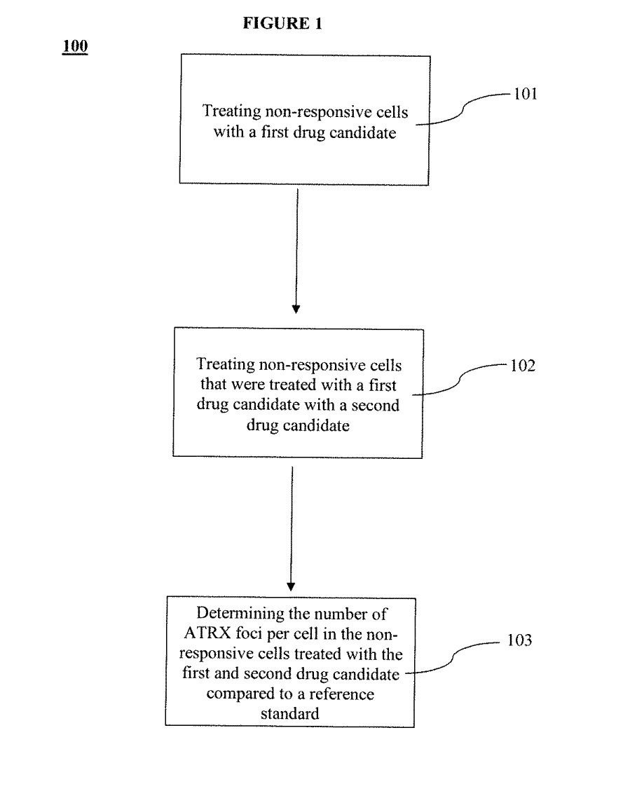

The disclosed assay of the present invention further provides a high-throughput screening method for identifying potential drug combinations that can be used to induce senescence in a cancer cell line. For purposes of illustration and not limitation, FIG. 1 is a schematic representation of an exemplary assay for identifying potential drug combinations according to the disclosed invention. In certain non-limiting embodiments, the assay of the present invention 100 includes treating one or more cells with a first drug candidate 101. The amount of the compound that is applied to the cells can depend on the type of compound used as the first drug candidate and the number of cells being treated. In non-limiting embodiments, cells can be treated with a first drug candidate at a concentration of about 1 nM to about 1 M. For example, and not by way of limitation, the cells can be treated with a first drug candidate at a concentration from about 10 nM to about 100 μM, from about 100 nM to about 10 μM, from about 500 nM to about 10 μM, from about 750 nM to about 10 μM, from about 750 nM to about 5 μM or from about 750 nM to about 1 μM. For example, and not by way of limitation, a first drug candidate can include PI3-kinase inhibitors and estrogen receptor antagonists.

Non-limiting examples of drug candidates that can be analyzed using the disclosed assays are described above. In certain embodiments, candidate compounds to be screened in the currently disclosed assay include known cancer chemotherapy agents such as, but not limited to, actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vinblastine, vincristine, vindesine and vinorelbine. In particular non-limiting embodiments, the candidate compound is not a CDK4 inhibitor. For example, and not by way of limitation, the first and/or second drug candidates is not a CDK4 inhibitor. In certain embodiments, the first candidate drug is not a CDK4 inhibitor. In certain embodiments, the second candidate drug is not a CDK4 inhibitor.

In non-limiting embodiments, cells for use in the disclosed assay can be a “non-responder cell.” In certain non-limiting embodiments, a non-responder cell is a cell that when treated with an amount of the first drug candidate effective for inducing senescence in a responder cell does not increase expression of at least one marker, or at least two markers, or at least three markers, of the senescent phenotype selected from the group consisting of senescence-associated beta-galactosidase (SA-β-gal), senescence-associated heterochromatin foci and elaboration of the senescence-associated secretory program and/or does not increase the number of ATRX foci in the cell, e.g., nucleus.

A “responder cell,” as used herein, refers to a cell that when treated with an effective amount of a drug candidate, e.g., a first drug candidate, or a compound that induces senescence, increases expression of at least one marker, or at least two markers, or at least three markers of the senescent phenotype selected from the group consisting of SA-β-gal, senescence-associated heterochromatin foci and elaboration of the senescence-associated secretory program and/or increases the number of ATRX foci in the nucleus. In non-limiting embodiments, the mean level of nuclear ATRX foci increase may be at least 30%.

In non-limiting embodiments, cells for use in the disclosed assay can be any cell line that undergoes quiescence in response to CDK4 inhibition but does not progress towards senescence as measured by accumulation of either SA-β-gal, senescence associated heterochromatic foci and/or elaboration of the senescence associated secretome. In certain non-limiting embodiments, a cell line that undergoes quiescence in response to CDK4 inhibition does not increase expression of at least one marker, or at least two markers, or at least three markers of the senescent phenotype selected from the group consisting of SA-β-gal, senescence-associated heterochromatin foci and elaboration of the senescence-associated secretory program and/or does not increase the number of ATRX foci in the nucleus and/or exhibits stable or increased levels of MDM2 protein, relative to the level without treatment with the CDK4 inhibitor.

In certain non-limiting embodiments, the cells for use in the disclosed assay can include LS8107, LS7785-1, LS7785-10, LS8313, H358 and H3122. In a non-limiting embodiment, the cells can be LS8107 cells. In non-limiting embodiments, the cells can be cancer cells from a patient, or a population of cells cultured from cancer cells from a patient.

In certain non-limiting embodiments, the cell lines used in the present assay can be a cancer cell line that does not undergo senescence in response to treatment with the compound used as the first drug candidate. For example, and not by way of limitation, a cell line that does not undergo senescence can be a cell line that does not increase expression of at least one marker, or at least two markers, or at least three markers of the senescent phenotype selected from the group consisting of SA-β-gal, senescence-associated heterochromatin foci and elaboration of the senescence-associated secretory program and/or does not increase the number of ATRX foci in the nucleus relative to the level observed in the absence of treatment with the first drug candidate.

After treatment with the first drug candidate, the one or more cells can subsequently be treated with a second drug candidate 102. Non-limiting examples of candidate compounds that can be used as the second drug candidate are disclosed above. In certain embodiments, the first drug candidate and the second drug candidate are different compounds. In certain embodiments, the cells can be treated with a second drug candidate at least one day, at least two days, at least three days, at least four days, at least five days, at least six days, at least seven days, at least eight days or more after treatment with the first drug candidate. In a specific non-limiting embodiment, the one or more cells can be treated with the second drug candidate two days after treatment with the first drug candidate. The amount of the second drug candidate applied to the cells depends on the type of compound used as the second drug candidate. In non-limiting embodiments, cells can be treated with a second drug candidate at a concentration of about 1 nM to about 1 M. For example, and not by way of limitation, the cells can be treated with a second drug candidate at a concentration from about 10 nM to about 100 μM, from about 100 nM to about 10 μM, from about 500 nM to about 10 μM, from about 750 nM to about 10 μM, from about 750 nM to about 5 μM or from about 750 nM to about 1 μM.

Following treatment with the second drug candidate, the assay method can further include determining the number of ATRX foci per cell 103, where an increase in the number of ATRX foci per cell in response to treatment with the second drug candidate indicates that the second drug candidate may be useful when administered in combination with the first drug candidate during the treatment of a subject that has cancer. In non-limiting embodiments, determining the number of ATRX foci can be performed at least two days, at least three days, at least four days, at least five days, at least six days, at least seven days, at least eight days or more after treatment with the second drug candidate.

In non-limiting embodiments, an increase in the number of ATRX foci may be appreciated by comparing the number of ATRX foci per cell in the non-responder cells following treatment with the second drug candidate to a reference standard. In certain, non-limiting embodiments, the reference standard can include non-responsive cells that have been treated with the first drug candidate alone. Alternatively or additionally, the reference standard can include non-responsive cells that have not been treated with a first drug candidate or a second drug candidate. In non-limiting embodiments, an increase in the percentage of cells that have a number of ATRX foci that increase about 50%, about 60%, about 70% or more per cell on average in response to treatment with both the first and second drug candidates as compared to a reference standard is indicative that the combination may be useful in treating subjects having cancer.

“In combination with,” “combinatorial” or “in conjunction with,” as used interchangeably herein, means that the first drug candidate and the second drug candidate are administered to a subject as part of a treatment regimen or plan. In certain embodiments, being used in combination does not require that the first drug candidate and the second drug candidate are physically combined prior to administration or that they be administered over the same time frame. For example, and not by way of limitation, the first drug candidate and the second drug candidate can be administered concurrently to the subject being treated, or can be administered at the same time or sequentially in any order or at different points in time.

### 5.2.2. Assay for Identifying Compounds that Prevent, Minimize, Inhibit and/or Reduce Senescence

The present invention further provides assays for identifying potential drug candidates that can be used to minimize, prevent, inhibit and/or reduce senescence in a cell line. In certain embodiments, the presently disclosed assays can be used to identify compounds that can prevent or inhibit a quiescent cell from transitioning to a senescent state and/or can result in a senescent cell to reenter the cell cycle and/or enter a quiescent state. The assays of the present invention comprise analyzing the number of ATRX foci per cell observed in the cell line following treatment with a drug candidate.

The present invention provides assays for identifying drug candidates that may be effective as therapeutic agents for treating diseases that are associated with senescence. For example, and not by way of limitation, the drug candidates may be effective as therapeutic agents for treating age-related diseases. Non-limiting examples of such diseases include heart disease, atherosclerosis, intervertebral disc degeneration, emphysema, glomerular disease, chronic obstructive pulmonary disease, skin aging, neurodegeneration, reduced organ function, e.g., liver, Alzheimer's, Parkinson's, arthritis, e.g., osteoarthritis, dementia and diabetes.

In certain embodiments, the assay for identifying compounds that can inhibit, minimize, prevent, inhibit and/or reduce senescence can include treating one or more cells with a drug candidate in combination with a compound or modality that induces senescence in the cells (i.e., a “senescence-inducing compound”). For example, and not by way of limitation, the senescence-inducing compound is a compound or modality that can result in an increase in the expression of at least one marker, or at least two markers, or at least three markers of the senescent phenotype selected from the group consisting of SA-β-gal, senescence-associated heterochromatin foci and elaboration of the senescence-associated secretory program and/or an increase the number of ATRX foci in the cell relative to the level observed in the absence of treatment with the compound or modality. In certain embodiments, the senescence-inducing compound can be doxorubicin. In certain embodiments, the senescence-inducing compound can be a CDK4 inhibitor, e.g., PD0332991. In certain embodiments, the senescence-inducing compound can be a modality that results in a reduction in MDM2 expression, e.g., siRNA or shRNA that targets MDM2. In non-limiting embodiments, the cells can be treated with the drug candidate and the senescence-inducing compound simultaneously. Alternatively, the cells can be treated with the drug candidate at least one day, at least two days, at least three days, at least four days, at least five days, at least six days, at least seven days, at least eight days or more after treatment with the senescence-inducing compound. Additionally, the cells can be treated with the drug candidate at least one day, at least two days, at least three days, at least four days, at least five days, at least six days, at least seven days, at least eight days or more before treatment of the cells with the senescence-inducing compound.

In certain embodiments, the assay can further include determining the number of ATRX foci per cell following treatment with the drug candidate. In certain embodiments, if a decrease in the number of ATRX foci per cell is observed or if the number of ATRX foci per cell does not increase in response to treatment with the drug candidate in combination with the senescence-inducing compound then the drug candidate may be useful in preventing and/or inhibiting the induction of senescence or reducing senescence and/or be useful as a therapy for treating age-related diseases.

In non-limiting embodiments, cells for use in the disclosed assay can be a cell that when treated with an effective amount of a senescence-inducing compound increases expression of at least one marker, or at least two markers, or at least three markers of the senescent phenotype selected from the group consisting of SA-β-gal, senescence-associated heterochromatin foci and elaboration of the senescence-associated secretory program and/or increases the number of ATRX foci in the nucleus. Non-limiting examples of such cells include LS8817, LS141 and LS0082. In non-limiting embodiments, the mean level of nuclear ATRX foci increase per cell may be at least 30%. In certain embodiments, cells for use in the disclosed assay can be a responder cell, as disclosed above.

In certain embodiments, the assay for identifying drug candidates that can minimize, prevent, inhibit and/or reduce senescence can comprise treating one or more cells that are in a senescent state, e.g., due to replicative senescence, with a drug candidate. In certain embodiments, senescence can be induced by the reduction in MDM2 expression in the cell. For example, and not by way of limitation, MDM2 expression can be reduced by the introduction of a modality that reduces MDM2 expression, e.g., a shRNA targeting MDM2 (i.e., shMDM2) or an siRNA targeting MDM2, into the cell, e.g., a LS8817 cell. In certain embodiments, the cell line can be the LS8313 cell line. In certain embodiments, the reduction in MDM2 expression can occur simultaneously with the treatment of the cells with the drug candidate. Alternatively, the cells can be treated with the drug candidate at least one day, at least two days, at least three days, at least four days, at least five days, at least six days, at least seven days, at least eight days or more after the reduction in MDM2 expression. In certain non-limiting embodiments, the assay can further include determining the number of ATRX foci per cell following treatment with the drug candidate where a decrease in the number of ATRX foci per cell in response to treatment with the drug candidate indicates that the drug candidate may be useful in transitioning the cells from a senescent state to a quiescent state and/or to induce the cells to reenter the cell cycle. In certain embodiments, the assay can further include determining whether the one or more cells reenter the cell cycle by counting the total number of cells, e.g., by DAPI staining.

The presently disclosed invention further provides assays for preventing, inhibiting, minimizing and/or reducing geroconversion in cells. As discussed in the Examples section below, geroconversion is the transition from a quiescent state to a senescence state and is associated with an increase in the number of ATRX foci in each cell. In certain embodiments, the assay for identifying drug candidates that can reduce, minimize, inhibit and/or prevent geroconversion, i.e., the transition from a quiescent state to a senescent state, can comprise treating one or more cells with a senescence-inducing compound, e.g., a CDK4 inhibitor, where the cell line undergoes quiescence in response to treatment with the senescence-inducing compound but does not progress towards senescence as measured by accumulation of either SA-β-gal, senescence associated heterochromatic foci and/or elaboration of the senescence associated secretome. In certain non-limiting embodiments, a cell line that undergoes quiescence in response to treatment with the senescence-inducing compound does not increase expression of at least one marker, or at least two markers, or at least three markers of the senescent phenotype selected from the group consisting of SA-β-gal, senescence-associated heterochromatin foci and elaboration of the senescence-associated secretory program and/or does not increase the number of ATRX foci in the nucleus and/or exhibits stable or increased levels of MDM2 protein, relative to the level without treatment with the compound. Non-limiting examples of such a cell line include LS8107, LS7785-1, LS7785-10, LS8313, H358 and H3122.

In certain embodiments, the assay can further include reducing MDM2 expression in the one or more senescence-inducing compound-treated cells in the presence of a drug candidate. In non-limiting embodiments, the reduction in MDM2 expression can occur simultaneously with the treatment of the cells with the drug candidate. Alternatively, the cells can be treated with the drug candidate at least one day, at least two days, at least three days, at least four days, at least five days, at least six days, at least seven days, at least eight days or more after the reduction in MDM2 expression. In certain embodiments, the cell line can comprise a modality that reduces MDM2 expression, e.g., a shRNA targeting MDM2 (i.e., shMDM2) or an siRNA targeting MDM2. In certain embodiments, the modality, e.g., shMDM2, can be under the control of tetracycline or its derivative doxycycline (e.g., tet-shMDM2), and the expression of the modality includes contacting the cells with doxycycline, e.g., to induce expression of the shMDM2. In certain embodiments, the assay can further include determining the number of ATRX foci per cell following treatment with the drug candidate, where a decrease in the number of ATRX foci per cell or where no increase in the number of ATRX foci per cell is observed in response to treatment with the drug candidate in the presence of the modality that targets MDM2 expression, as compared to a reference standard, indicates that the drug candidate may be useful in preventing the induction of senescence or reducing senescence.

In certain embodiments, the assay for identifying drug candidates that can reduce, minimize, inhibit and/or prevent geroconversion, can comprise treating one or more cells with a senescence-inducing compound, e.g., a CDK4 inhibitor, in the presence of exogenously-expressed MDM2, e.g., human MDM2, where the cell line undergoes quiescence in response to treatment with the senescence-inducing compound and MDM2 expression but does not progress towards senescence. Non-limiting examples of such a cell line for use in this assay includes LS8817, LS141 and LS0082 cell lines. In certain embodiments, the cell line can comprise a modality that increases MDM2 expression, e.g., a construct that comprises full-length MDM2. In certain embodiments, the exogenously-expressed MDM2 can be tagged, for example, with a Flag-tag, a Myc-tag or an HA-tag. In certain embodiments, the modality can be under the control of tetracycline or its derivative doxycycline (e.g., tet-MDM2), and the expression of MDM2 is obtained by contacting the cells with doxycycline. In certain embodiments, the assay can further include the removal of doxycycline from the cells and the treatment of the cells with a drug candidate. In non-limiting embodiments, the removal of doxycycline can occur simultaneously with the treatment of the cells with the drug candidate. Alternatively, the cells can be treated with the drug candidate at least one day, at least two days, at least three days, at least four days, at least five days, at least six days, at least seven days, at least eight days or more after the removal of doxycycline. In certain embodiments, the cells can be treated with the drug candidate at least one day, at least two days, at least three days, at least four days, at least five days, at least six days, at least seven days, at least eight days or more prior to removal of doxycycline from the cells. In certain embodiments, the assay can further include determining the number of ATRX foci per cell following treatment with the drug candidate and the removal of doxycycline, where a decrease in the number of ATRX foci per cell or where no increase in the number of ATRX foci per cell is observed in response to treatment with the drug candidate in the absence of doxycycline, as compared to a reference standard, indicates that the drug candidate may be useful in preventing and/or inhibiting the induction of senescence or reducing senescence.

In non-limiting embodiments, determining the number of ATRX foci can be performed within at least two days, at least three days, at least four days, at least five days, at least six days, at least seven days, at least eight days or more after treatment with the drug candidate. In non-limiting embodiments, a decrease in the number of ATRX foci or the lack of an increase in the number of ATRX foci may be appreciated by comparing the number of ATRX foci per cell in the cells following treatment with the drug candidate to a reference standard. In certain, non-limiting embodiments, the reference standard can include cells that have been treated with the senescence-inducing compound alone. Alternatively or additionally, the reference standard can include senescence cells that have not been treated with the drug candidate or senescence-inducing compound. In certain embodiments, the reference standard can include cells that have been treated with a senescence-inducing compound followed by treatment with a modality that reduces MDM2 expression. In certain embodiments, the reference standard can include cells that have been treated with a senescence-inducing compound in the presence of exogenously expressed MDM2. In non-limiting embodiments, a decrease in the percentage of cells or no increase in the percentage of cells that have an increased number of ATRX foci per cell in response to treatment with the drug candidate as compared to a reference standard is indicative that the drug candidate may be useful in treating subjects having an age-related disease. In certain embodiments, an increase in the average number of ATRX foci per cell of less than about 50%, less than about 45%, less than about 40%, less than about 35%, less than about 30%, less than about 25%, less than about 20%, less than about 15%, less than about 10%, less than about 5%, less than about 2% or less than about 1% upon treatment with the drug candidate, e.g., as compared to a reference standard, is indicative that the drug candidate may be useful in treating subjects having an age-related disease.

The amount of the drug candidate that is applied to the cells can depend on the type of compound used as the drug candidate and the number of cells being treated. Non-limiting examples of drug candidates or candidate compound libraries that can be analyzed using the disclosed assays are discussed above. In certain non-limiting embodiments, cells can be treated with a drug candidate at a concentration of about 1 nM to about 1 M. For example, and not by way of limitation, the cells can be treated with the drug candidate at a concentration from about 10 nM to about 100 μM, from about 100 nM to about 10 mM, from about 500 nM to about 10 μM, from about 750 nM to about 10 μM, from about 750 nM to about 5 μM or from about 750 nM to about 1 μM.

The amount of the senescence-inducing compound that is applied to the cells can depend on the type of compound used as the senescence-inducing compound and the number of cells being treated. Non-limiting examples of senescence-inducing compounds that can be used in the disclosed assays are discussed above. In certain non-limiting embodiments, cells can be treated with a senescence-inducing compound at a concentration of about 1 nM to about 1 M. For example, and not by way of limitation, the cells can be treated with the senescence-inducing compound at a concentration from about 10 nM to about 100 μM, from about 100 nM to about 10 μM, from about 500 nM to about 10 μM, from about 750 nM to about 10 μM, from about 750 nM to about 5 μM or from about 750 nM to about 1 μM. In certain embodiments, the senescence-inducing compound can be applied to the cells at a concentration of about 1 μM. In certain embodiments, the senescence-inducing compound can be applied to the cells at a concentration of about 0.1 μM.

### 5.3 Kits

In non-limiting embodiments, the present invention provides for a kit for determining whether a drug combination induces senescence and/or whether a drug candidate prevents, minimizes, inhibits and/or reduces senescence in a cell, comprising a means for detecting ATRX foci. Methods for measuring ATRX foci are described in the sections above.

Types of kits include, but are not limited to, arrays/microarrays, ATRX-specific, antibodies or other detection reagents for detecting ATRX foci.

In non-limiting embodiments, a kit may comprise at least one antibody for immunodetecting ATRX foci. In one specific non-limiting embodiment, a kit may comprise a probe or antibody suitable for detecting ATRX protein present within the foci. Antibodies, both polyclonal and monoclonal, including molecules comprising an antibody variable region or a subregion thereof, specific for an ATRX protein, may be prepared using conventional immunization techniques, as will be generally known to those of skill in the art. In non-limiting embodiments, a kit of the present invention can comprise the ATRX-specific antibody sold by Bethyl, Catalog No. A301-045A (“the '045 Ab”), a fragment thereof, or an antibody that competitively inhibits binding of the '045 Ab to ATRX, for detecting ATRX foci. In certain embodiments, a kit of the present invention can comprise the ATRX-specific antibody sold by Santa Cruz Biotechnology, Catalog No. sc-55584 (the “D5 antibody”), a fragment thereof, or an antibody that competitively inhibits binding of the D5 antibody to ATRX, for detecting ATRX foci.

The immunodetection reagents of the kit may include detectable labels that are associated with, or linked to, the given antibody or antigen itself. Such detectable labels include, for example, fluorescent molecules (rhodamine, fluorescein, green fluorescent protein, luciferase, Cy3, Cy5, or ROX), radiolabels (3H, 35S, 32P, 14C, 131I) or enzymes (alkaline phosphatase, horseradish peroxidase). Alternatively, a detectable moiety may be comprised in a secondary antibody or antibody fragment, which selectively binds to the first antibody or antibody fragment (where said first antibody or antibody fragment specifically recognizes ATRX).

In certain non-limiting embodiments, a kit may comprise one or more detection reagents and other components (e.g., a buffer, enzymes such as alkaline phosphatase, antibodies, and the like) necessary to carry out an assay or reaction to determine the number of ATRX foci per cell.

In a further non-limiting embodiment, the kit may further include one or more cells for use in the disclosed assay. Non-limiting examples of cells for use in the disclosed assays are described above. For example, and not by way of limitation, the kit can include LS8107 cells. In certain embodiments, the kit can include LS8107 or LS8313 cells that comprise a vector comprising a shMDM2 under the control of doxycycline. In certain embodiments, the kit can include LS8817 cells. In certain embodiments, the kit can include LS8313 cells. In certain embodiments, the kit can include LS8817 cells that comprise a vector comprising a shMDM2 under the control of doxycycline. In certain embodiments, the kit can include LS8817 cells that comprise a vector comprising MDM2 (e.g., full-length MDM2) under the control of doxycycline. The cells within the kit can be supplied as a cell suspension or as a frozen cell sample. In non-limiting embodiments, the cells can be provided in a multiwell plate, a microtiter plate or in an array.

A kit may further include instructions for using the kit to determine the number of ATRX foci. In non-limiting embodiments, the instructions describe that an increase in the number of ATRX foci per cell upon treatment with the drug combination is indicative that the drug combination may be useful as a combinatorial drug therapy for cancer. Alternatively or additionally, in certain non-limiting embodiments, the instructions can describe that a decrease or the absence of an increase in the number of ATRX foci per cell upon treatment with a drug candidate is indicative that the drug candidate may be useful as a therapy for treating age-related diseases.

### 6. EXAMPLE 1

### Atrx Foci as a Marker for Senescence

6.1 Materials and Methods

Senescence Analyses.

Cells were plated at a concentration of 25,000 per well in a 4-well chamber slides (Lab-Tek) and treated for seven days with drug and stained for senescence-associated β-galactosidase (Cell Signaling kit #9860). Cell number was quantitated by DAPI staining and β-galactosidase staining quantitated as a proportion of total cells.

Immunofluorescence.

Cells were washed twice with PBS and fixed with 4% paraformaldehyde at room temperature for 20 minutes. Cells were again washed twice with PBS and treated with 0.5% triton X-100 in PBS. Cells were again washed twice with PBS and blocked in 5% BSA in PBS for 30 minutes at room temperature. Cells were incubated with anti-ATRX antibody, obtained from Bethyl, Cat No. A301-045A, diluted at 1:2000 in PBS (stock is provided at 1 mg/ml) overnight at 4 degrees Celsius. The following day, cells were washed twice with PBS and incubated with Alexa Fluor-488 anti-rabbit secondary diluted at 1:500 in PBS (stock was provided at 2 mg/ml) for one hour at room temperature. Cells were washed twice with PBS and incubated with 0.1 μg/ml DAPI for 5 minutes at room temperature. Cells were washed three times with PBS and slides were mounted in Vectashield fluorescence mounting media. Slides were imaged using a Zeiss Axioplan 2 Upright Microscope.

Immunoblot.

Antibodies against p53 (Bp53-12), were obtained from Santa Cruz Biotechnology, and the ATRX antibody, Cat No. A301-045A was obtained from Bethyl. Treated cells were lysed with buffer composed of 50 mM Tris-HCl, pH7.4, 250 mM NaCl, 5 mM EDTA, 0.5% NP40, 2 mM PMSF, and supplemented with protease inhibitors. Eighty micrograms of protein were resolved by SDS-PAGE and transferred to PVDF membranes. Membranes were incubated overnight with antibodies (1:1000).

### 6.2 Results

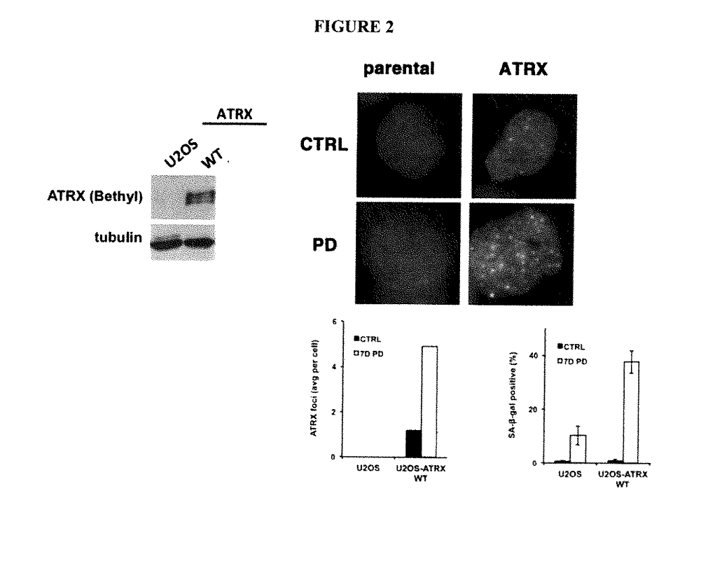

Senescence is perceived as a favorable clinical outcome due to its ability to inhibit tumor progression. ATRX plays a role in senescence and has been shown to interact with PML, macroH2A, HP1 and histone H3.3 (see Eustermann et al., 2011; Lewis et al., 2010; Ratnakumar et al., 2012; Xue et al., 2003), which have been show to interact with HIRA/ASF. Immunofluorescence analysis using the Bethyl antibody was performed to determine if ATRX was recruited to foci. As shown in FIG. 2, ATRX is required for senescence induced by the inhibition of CDK4. In U2OS cells that do not express endogenous ATRX, inhibition of CDK4 using the CDK4 inhibitor, PD 0332991, resulted in the cells entering a quiescent state versus a senescent state (FIG. 2). Expression of exogenous ATRX in U2OS cells, in the presence of the CDK4 inhibitor, resulted in the cells entering a senescent state as determined by the accumulation of the senescence marker, perinuclear associated β-galactosidase (SA-β-gal). The exogenous expression of a mutant form of ATRX in the U2OS cells did not modulate the response to CDK4 inhibition.

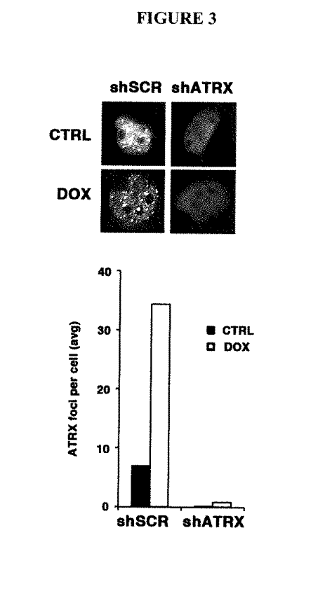

As shown in FIG. 3, ATRX foci were observed upon treatment with doxorubicin. LS8817 cells expressing either a scrambled (shSCR) or ATRX shRNA (shATRX) were treated with 100 μM doxorubicin for five days. In doxorubicin treated cells, the number of ATRX foci per cell significantly increased compared to cells that were not treated with doxorubicin (FIG. 3). As shown in FIG. 3, the average number of ATRX foci per cell in doxorubicin-treated cells was about 35; whereas, the average number of ATRX foci per cell in the control cells was about 8. In addition, ATRX foci were not observed in cells expressing shATRX (FIG. 3).

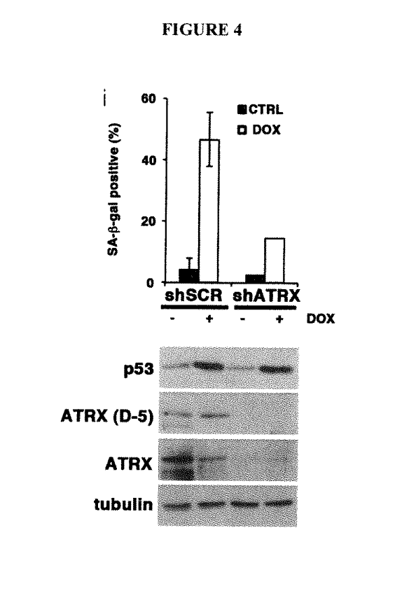

To determine if doxorubicin induced senescence in the liposarcoma cell line, LS8817, the accumulation of SA-3-gal was measured following treatment with doxorubicin (FIG. 4). As shown in FIG. 4, LS8817 cells expressing either a shSCR or shATRX were treated with 100 μM doxorubicin for five days and the effect on the accumulation of SA-β-gal was determined (bottom). SA-β-gal staining significantly accumulated after doxorubicin treatment in cells expressing shSCR. In contrast, SA-β-gal staining did not significantly accumulate in shATRX-expressing cells after doxorubicin treatment (FIG. 4).

6.1 Discussion

Without being bound to a particular theory, these results indicate that senescence, regardless of the nature of the inducer, is correlated with an increase in the number of ATRX foci in each cell, and disruption of ATRX prevents senescence. Therefore, ATRX is generally required for senescence, and this is associated with its recruitment to chromatin-associated foci.

### 7. EXAMPLE 2

### Atrx Foci as a Marker for Replicative Senescence and Geroconversion

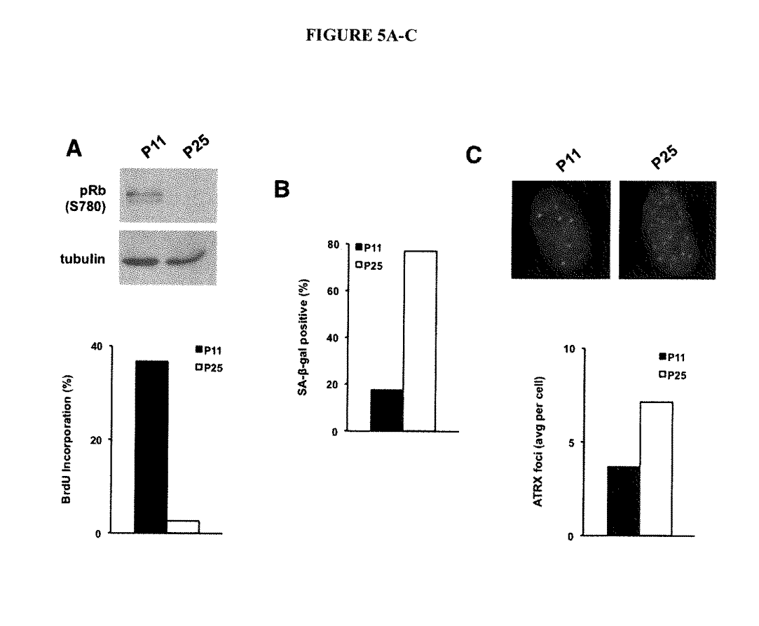

To determine whether ATRX foci are observed upon induction of senescence in the untransformed cell line, WI-38, the cells were continually passaged until passage number 25 to induce replicative senescence. As the cells were continually passaged to passage 25, cell proliferation was observed to have greatly reduced as determined by BrdU staining. In addition, the expression of p53, which has been previously shown to increase in expression in proliferating cells, was greatly reduced in cells at passage 25 (FIG. 5A).

To determine if continual passaging of the WI-38 cell line induced senescence, the accumulation of SA-β-gal was measured during passage 11 and passage 25. As shown in FIG. 5B, SA-β-gal staining significantly accumulated after passage 25 as compared to passage 11, suggesting that the cells under senescence during continual passaging. In addition, the number of ATRX foci per cell significantly increased at passage 25 as compared to cells at passage 11 (FIG. 5C). These results show that replicative senescence is associated with an increase in ATRX foci, and ATRX foci can be a marker for normal, untransformed cells undergoing senescence.

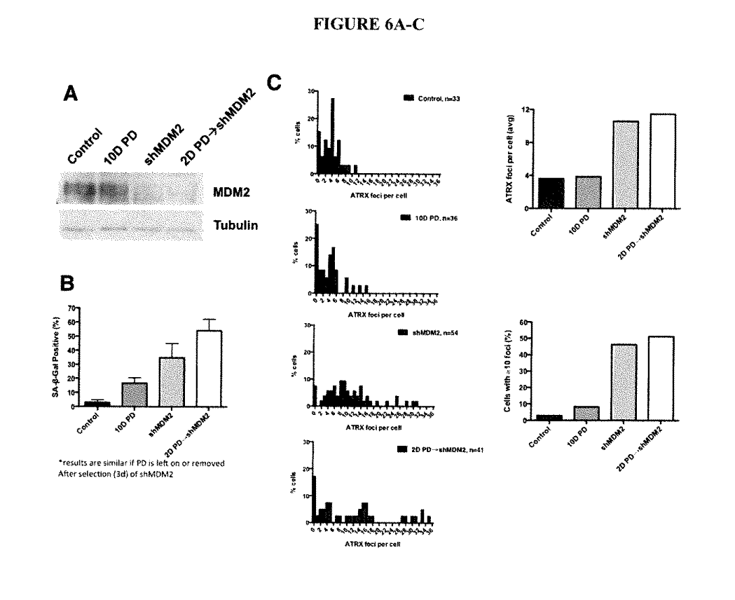

In addition, an increase in the number of ATRX foci was observed during geroconversion, which is the transition from a quiescent state to a senescence state. As shown in FIG. 6, LS8107 cells, a cancer cell line that does not undergo senescence in response to treatment with the CDK4 inhibitor, PD0332991, entered a quiescent state in the presence of PD032991. Following treatment with PD032991, the cells were than treated with a shRNA directed to MDM2 to decrease MDM2 expression (FIG. 6A). As shown in FIG. 6, the loss of MDM2 expression following CDK4 inhibition resulted in the cells transitioning from a quiescent state to a senescent state as indicated by the accumulation of SA-β-gal staining (FIG. 6B) and resulted in an increase in the number of ATRX foci per cell as compared to the cells treated with PD0332991 alone (FIG. 6C). Without being bound to a particular theory, these results indicate that geroconversion is correlated with an increase in the number of ATRX foci in each cell.

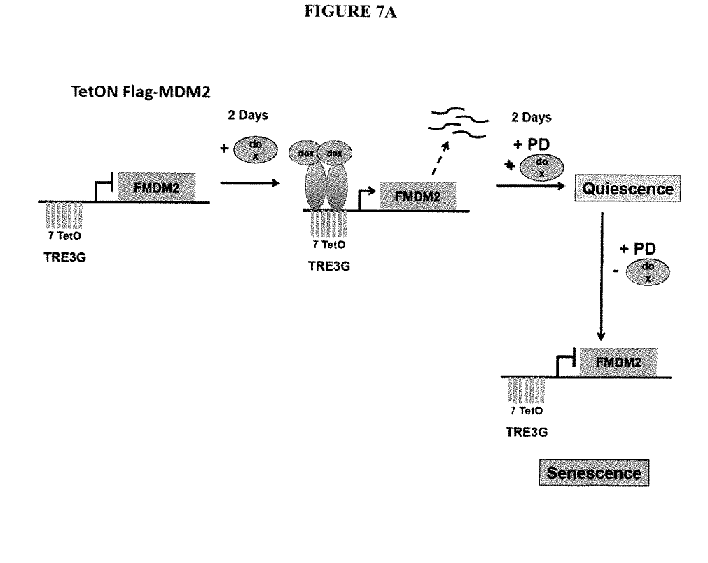

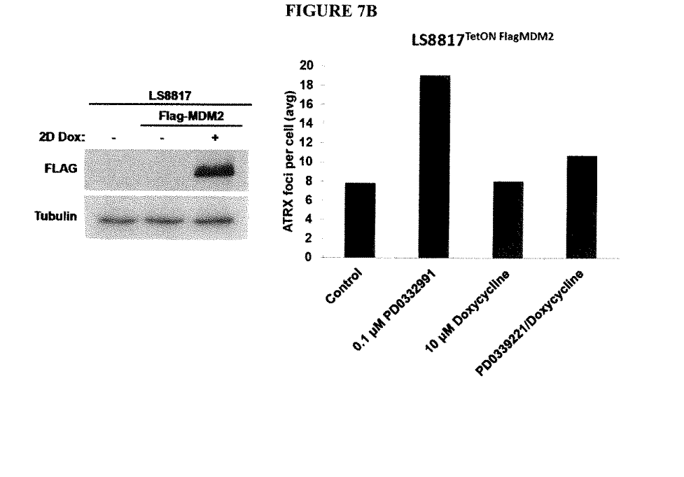

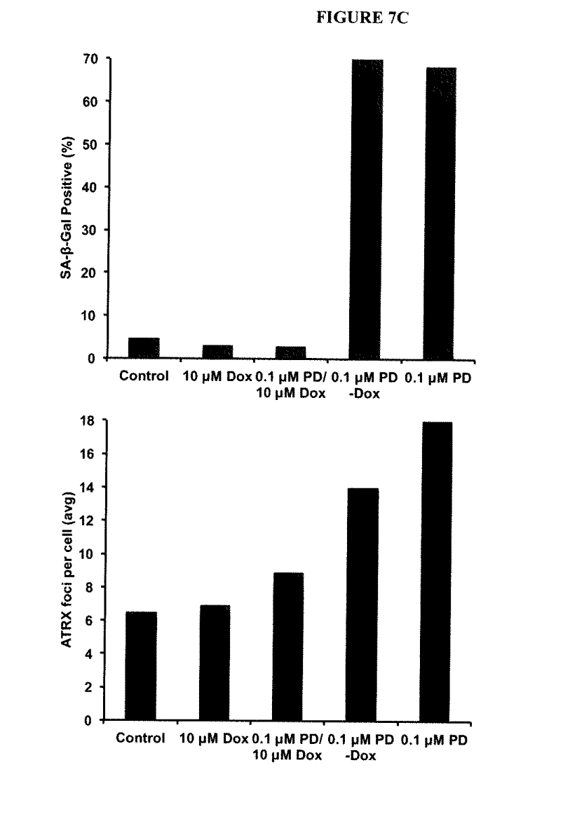

In the liposarcoma cell line, LS8817, the CDK4 inhibitor, PD0332991, at a concentration of 1 μM or 0.1 μM, resulted in the cells to enter a senescent state. This senescent state correlated with an significant increase in the average number of ATRX foci per cell (FIG. 7B). The exogenous expression of MDM2 by the use of a tet-on-MDM2-Flag construct within these CDK4 inhibitor-treated cells in the presence of doxycycline resulted in the cells to enter a quiescent state and prevented them from undergoing senescence (FIGS. 7A and 7C). The removal of doxycycline allowed the cells to enter senescence and resulted in an increase in the average number of ATRX foci per cell (FIGS. 7A and 7C). In addition, as shown in FIG. 7C, the removal of doxycycline to reduce exogenous MDM2 expression allowed the cells to enter a senescent state as indicated by the accumulation of SA-β-gal staining.

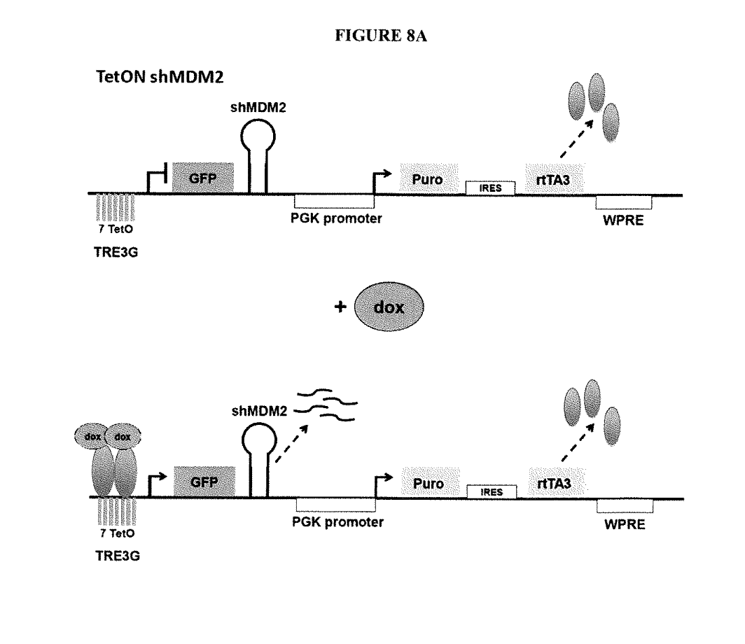

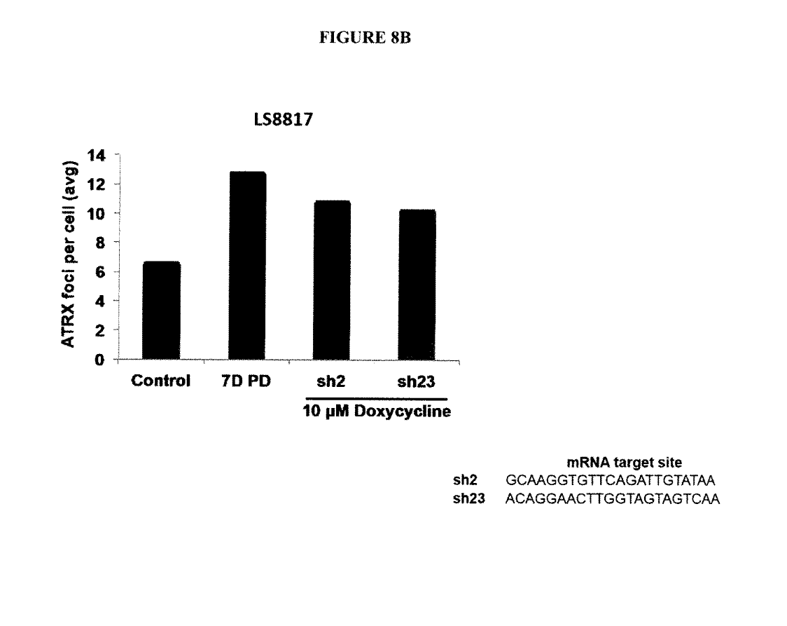

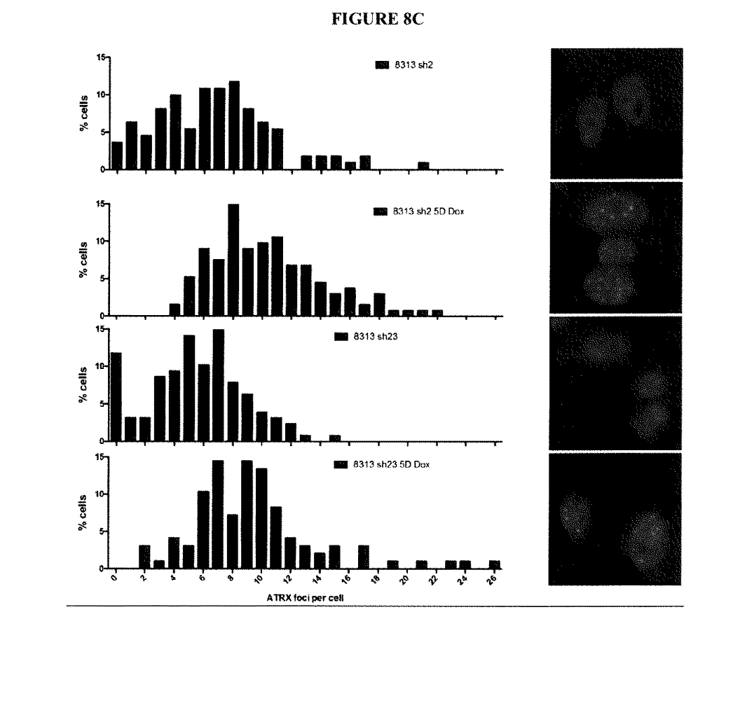

To determine if the reduction in MDM2 alone can result in the induction of senescence, LS8817 cells or LS8313 cells, which included a tet-on-shMDM2 construct, were treated with doxycycline to express shMDM2 (FIG. 8A). The target of the shMDM2 constructs as shown in FIG. 8B. As shown in FIG. 8B, the reduction in the expression of MDM2 alone in LS8817 cells resulted in the cells entering a senescent state. The number of ATRX foci also increased upon the reduction in MDM2 expression (FIG. 8B). In addition, and as shown in FIG. 8C, the reduction of MDM2 expression alone in LS8313 cells was sufficient to induce the cells to enter a senescent state as indicated by the increase in the number of ATRX foci observed per cell as compared to the control.

Various references are cited herein, the contents of which are hereby incorporated by reference in their entireties.

What is claimed is:

1. An assay for identifying drug candidates for use in a combinatorial cancer treatment, comprising:
(a) treating one or more cells with a first drug candidate;
(b) treating the one or more first drug candidate-treated cells with a second drug candidate; and
(c) determining the number of ATRX foci per cell in the one or more first and second drug candidate-treated cells, as compared to a reference, where an increase in the number of ATRX foci per cell compared to the reference is indicative that the combination of the first drug and the second drug is likely to be useful as a combinatorial cancer drug therapy.

2. The assay of claim 1, wherein the number of ATRX foci per cell is determined by immunofluorescence.

3. The assay of claim 1, wherein the one or more cells is LS8107.

4. The assay of claim 1, wherein the one or more cells is a cell that is not responsive to treatment with the first drug candidate.

5. The assay of claim 1, wherein the drug candidates are selected from the group consisting of small chemical molecules, biologics, and peptides.

6. The assay of claim 1, wherein the ATRX foci is detected by an ATRX-specific antibody.

7. The assay of claim 6, wherein the ATRX-specific antibody is the antibody sold by Bethyl, Catalog No. A301-045A (“the '045 Ab”), a fragment thereof (e.g., a Fab fragment, a Fab₂, or the variable region comprised in a chimeric molecule) or an antibody that competitively inhibits binding of the '045 Ab to ATRX.

8. The assay of claim 1, wherein the reference sample is one or more cells treated with the first drug candidate alone.

9. An assay for identifying drug candidates for use in a combinatorial cancer drug treatment, comprising:
(a) treating one or more non-responsive cells with a first drug candidate;
(b) treating the one or more first drug candidate-treated cells with a second drug candidate;
(c) determining the number of ATRX foci per cell in the one or more cells treated with the first and second drug candidates; and
(d) comparing the number of ATRX foci in the one or more cells treated with the first and second drug candidates to a reference,
where an increase in the number of ATRX foci per cell in the first and second compound-treated cells compared to the reference is indicative that the combination of the first drug and the second drug is likely to be useful as a combinatorial cancer drug therapy.

10. The assay of claim 9, wherein the number of ATRX foci per cell is determined by immunofluorescence.

11. The assay of claim 9, wherein the non-responsive cell is LS8107.

12. The assay of claim 9, wherein the non-responsive cell is a cell that is not responsive to treatment with the first drug candidate.

13. The assay of claim 9, wherein the drug candidates are selected from the group consisting of small chemical molecules, biologics, and peptides.

14. The assay of claim 9, wherein the ATRX foci is detected by an ATRX-specific antibody.

15. The assay of claim 14, wherein the ATRX-specific antibody is the antibody sold by Bethyl, Catalog No. A301-045A (“the '045 Ab”), a fragment thereof (e.g., a Fab fragment, a Fab₂, or the variable region comprised in a chimeric molecule) or an antibody that competitively inhibits binding of the '045 Ab to ATRX;

16. The assay of claim 9, wherein the one or more first compound-treated cells is treated with the second drug candidate at least two days after treatment with the first drug candidate.

17. The assay of claim 9, wherein the reference sample is one or more non-responsive cells treated with the first drug candidate alone.

18. An assay for identifying drug candidates for use in treating an age-related disease, comprising:
(a) treating one or more cells with a compound that induces senescence;
(b) treating the one or more cells with a drug candidate; and
(c) determining the number of ATRX foci per cell in the compound and drug candidate-treated cells, as compared to a reference, where a decrease in the number of ATRX foci per cell or the absence of an increase in the number of ATRX foci per cell compared to the reference is indicative that the drug candidate is likely to be useful as a therapy for treating an age-related disease.

19. The assay of claim 18, wherein the number of ATRX foci per cell is determined by immunofluorescence.

20. The assay of claim 18, wherein the one or more cells is LS8817.

21. The assay of claim 18, wherein the compound that induces senescence and the drug candidate are added to the one or more cells simultaneously.

22. The assay of claim 18, wherein the compound that induces senescence is a CDK4 inhibitor.

23. The assay of claim 18, wherein the drug candidates are selected from the group consisting of small chemical molecules, biologics, and peptides.

24. The assay of claim 18, wherein the ATRX foci is detected by an ATRX-specific antibody.

25. The assay of claim 24, wherein the ATRX-specific antibody is the antibody sold by Bethyl, Catalog No. A301-045A (“the '045 Ab”), a fragment thereof (e.g., a Fab fragment, a Fab₂, or the variable region comprised in a chimeric molecule) or an antibody that competitively inhibits binding of the '045 Ab to ATRX.

26. The assay of claim 18, wherein the reference sample is one or more cells treated with the compound that induces senescence alone.

27. An assay for identifying drug candidates for use in treating an age-related disease, comprising:
(a) treating one or more cells with a CDK4 inhibitor, where the cells enter a quiescent state upon treatment with the compound that induces senescence.
(b) treating the one or more CDK4-inhibitor treated cells with shMDM2;
(c) treating the one or more cells with a drug candidate; and
(d) determining the number of ATRX foci per cell in the CDK4 inhibitor, shMDM2 and drug candidate-treated cells, as compared to a reference, where a decrease in the number of ATRX foci per cell or the absence of an increase in the number of ATRX foci per cell compared to the reference is indicative that the drug candidate is likely to be useful as a therapy for treating age-related disease.

28. The assay of claim 27, wherein the number of ATRX foci per cell is determined by immunofluorescence.

29. The assay of claim 27, wherein the one or more cells is LS8107.

30. The assay of claim 27, wherein the shMDM2 is expressed from a vector present in the one or more cells under the control of doxycycline;

31. The assay of claim 27, wherein the CDK4 inhibitor is PD0332991.

32. The assay of claim 27, wherein the drug candidate is selected from the group consisting of small chemical molecules, biologics, and peptides.

33. The assay of claim 27, wherein the ATRX foci is detected by an ATRX-specific antibody.

34. The assay of claim 33, wherein the ATRX-specific antibody is the antibody sold by Bethyl, Catalog No. A301-045A (“the '045 Ab”), a fragment thereof (e.g., a Fab fragment, a Fab₂, or the variable region comprised in a chimeric molecule) or an antibody that competitively inhibits binding of the '045 Ab to ATRX.

35. The assay of claim 27, wherein the reference sample is one or more cells treated with the CDK4 inhibitor and the shMDM2 in the absence of the drug candidate.
